(19) United States
(12) Patent Application Publication
(10) Pub. No.: US 2021/0056639 A1
(43) Pub. Date: Feb. 25, 2021

(54) TELEMEDICINE PRACTITIONER ONBOARDING WITH ON-DEMAND PER ENCOUNTER MALPRACTICE LIABILITY COVERAGE

(71) Applicant: Azova, Inc., Alpine, UT (US)

(72) Inventor: Cheryl Lee Eberting, Alpine, UT (US)

(21) Appl. No.: 17/000,138

(22) Filed: Aug. 21, 2020

Related U.S. Application Data
(60) Provisional application No. 62/890,610, filed on Aug. 22, 2019.

(51) Int. Cl. G06Q 40/08; G06Q 50/22; G16H 40/67

(52) U.S. Cl. CPC G06Q 40/08; G16H 40/67; G06Q 50/22

(57) ABSTRACT

Systems and methods are described herein for offering, providing, and charging for on-demand per-encounter liability coverage for healthcare practitioners engaged in telemedicine consultations in connection with a telemedicine platform. The per-encounter liability coverage may be provided to supplement an existing insurance policy of the healthcare practitioner that is, for example, limited to cover only in-person consultations. The on-demand per-encounter liability coverage may be offered in connection with enrollment by the healthcare practitioner in a telemedicine platform that facilitates various types of telemedicine consultations.

### RELATED APPLICATIONS

This application claims priority to U.S. Provisional Patent Application No. 62/890,610, filed on Aug. 22, 2019 and titled “TELEMEDICINE PRACTITIONER ONBOARDING WITH ON-DEMAND PER-ENCOUNTER MALPRACTICE LIABILITY COVERAGE,” which is hereby incorporated by reference in its entirety. This application is also related to PCT Application No. PCT/US2016/020964, filed Mar. 4, 2016, titled “TELEMEDICINE PLATFORM WITH INTEGRATED E-COMMERCE AND THIRD-PARTY INTERFACES,” which is also hereby incorporated by reference in its entirety.

### TECHNICAL FIELD

This disclosure relates to telemedicine platforms and associated services, including electronic medical record management, physician-customizable online portals, patient services, and health-related online marketplaces.

### BRIEF DESCRIPTION OF THE DRAWINGS

Non-limiting and non-exhaustive embodiments of the disclosure are described herein, including various embodiments of the disclosure illustrated in the figures listed below.

FIG. 1 illustrates an example of an enrollment system to enroll a healthcare practitioner in a telemedicine platform with on-demand per-encounter malpractice insurance coverage.

FIG. 2 illustrates a flow chart of a method to enroll a healthcare practitioner in a telemedicine platform, including enrollment in an on-demand per-encounter malpractice insurance coverage program.

FIG. 3 illustrates an embodiment of an example graphical user interface of an enrollment system to facilitate enrollment in a telemedicine healthcare marketplace plan of a telemedicine platform.

FIG. 4 illustrates an embodiment of an example graphical user interface for collecting enrollment information, including payment information.

FIG. 5 illustrates an embodiment of an example graphical user interface for creating a username and password for future logins into the telemedicine platform.

FIG. 6 illustrates an embodiment of an example graphical user interface for a healthcare practitioner to provide license information for validation by the enrollment system.

FIG. 7 illustrates an embodiment of an example graphical user interface of the enrollment system to facilitate the selection of services to be offered by the healthcare practitioner via the telemedicine platform.

FIG. 8 illustrates an embodiment of an example graphical user interface of the enrollment system to facilitate the selection of additional services to be offered by the healthcare practitioner via the telemedicine platform.

FIG. 9 illustrates an embodiment of an example graphical user interface of the enrollment system to facilitate the selection of additional services to be offered by the healthcare practitioner via the telemedicine platform.

FIG. 10 illustrates an embodiment of an example graphical user interface of the enrollment system to enable the healthcare practitioner to join a telemedicine provider network of the telemedicine platform.

FIG. 11 an embodiment of an example graphical user interface of the enrollment system to enable the healthcare practitioner to join a volunteer provider network of the telemedicine platform.

FIG. 12 illustrates an embodiment of an example graphical user interface of the enrollment system to receive state licenses and malpractice insurance information.

FIG. 13 illustrates an embodiment of an example graphical user interface of the enrollment system to prompt the healthcare practitioner to enter existing malpractice insurance information.

FIG. 14 illustrates an embodiment of an example graphical user interface of the enrollment system to prompt the healthcare practitioner to enter state license information for validation by the enrollment system.

FIG. 15 illustrates an embodiment of an example graphical user interface of the enrollment system to invite the healthcare practitioner to enroll or apply for malpractice insurance.

FIG. 16 illustrates an embodiment of an example graphical user interface of the enrollment system requesting information as part of a malpractice insurance application.

FIG. 17 illustrates an embodiment of an example graphical user interface of the enrollment system confirming enrollment.

### DETAILED DESCRIPTION

A telemedicine platform may include any number of features and options that may be commonly used by all healthcare practitioners and/or patients and other features and options that are not used by certain subsets of healthcare practitioners and/or patients, whether due to personal preference, usability, cost, or inapplicability. Accordingly, the present systems and methods provide various aspects, features, services, and functions relating to telemedicine healthcare platforms and auxiliary services and products. It is appreciated that any of the various embodiments described herein may be combined in any number of ways and that all permutations and combinations of the described features, advantages, embodiments, and options are part of this disclosure even if such permutations and combinations are not explicitly described in a single embodiment.

Healthcare practitioners may elect to provide a wide variety of healthcare services, features, functions, databases, subsystems, etc. via a telemedicine platform. The enrollment system described herein facilitates the selection of such services, validates licensure information, validates malpractice insurance information, and/or facilitates the enrollment in on-demand per-consultation malpractice insurance. The on-demand per-consultation malpractice insurance may be the only malpractice insurance the healthcare practitioner has, replace an existing malpractice insurance policy of the healthcare practitioner, or supplement an existing malpractice insurance policy of the healthcare practitioner.

The on-demand per-consultation malpractice insurance may be on-demand in the sense that it is available for purchase by a healthcare practitioner in real-time as the practitioner enrolls in the telemedicine platform and/or because the malpractice insurance is only applicable to consultations/visits conducted through the telemedicine platform associated with the enrollment system. That is, the on-demand per-consultation malpractice insurance does not cover the healthcare practitioner's normal, in-office consultations and visits. In various embodiments, the healthcare practitioner may be billed for the malpractice insurance on a per-consultation basis.

For example, the healthcare practitioner may have a malpractice insurance policy that covers her normal medical practice, including in-office visits. The healthcare practitioner may be interested in providing telemedicine services even though her existing malpractice insurance policy does not cover telemedicine visits and/or would incur additional expenses and/or be difficult or troublesome to update. The enrollment systems and methods described herein allow the healthcare practitioner to, for example, enroll in supplemental on-demand per-consultation malpractice insurance that will provide liability and/or other insurance coverage for the healthcare practitioner's telemedicine consultations conducted via one specific telemedicine platform or a set of approved telemedicine platforms.

The healthcare practitioner may pay for the on-demand per-consultation insurance on a per-visit basis or on a periodic basis associated with the number of actual visits, actual patients, actual consultation types, and/or prescriptions written. For example, the practitioner may be billed $1 per visit, $10 per patient, $100 for the first 200 patients, $1 per prescription written, $20 per day, and/or combinations thereof on a one-time or periodic basis.

The various insurance policy options may be fully or partially customized based on the particular healthcare practitioner's practice and/or the specific services selected to be offered via the telemedicine platform by the healthcare practitioner. Any of a wide variety of health and wellness platforms and/or services may be integrated as part of the enrollment process offered by the enrollment system

Examples of healthcare practitioners include, but are not limited to: pharmacists, physicians (MDs), osteopathic physicians (DOs), nurse practitioners, physician's assistants, mental health professions, psychologists, social workers, mental health therapists, health and wellness professionals, dieticians, nutritionists, associated insurers, agents, billing specialists, and/or other persons or entities associated with mental, beauty, physical, and other healthcare areas.

Many of the embodiments described herein use a “healthcare practitioner” or just a “practitioner” as an example of the entity (e.g., person, persons, or business) that is utilizing the enrollment system. However, it is appreciated that the entity actually managing, setting up, initializing, or otherwise involved with the enrollment system may be a healthcare administrator, information technology (IT) specialist, or another entity that does not necessarily treat, test, diagnose, or otherwise interface with patients. Such entities may be referred to as “providers” inasmuch as they act as the agent of the healthcare provider that actually provides the services to patients.

Software offerings available may include software (e.g., computer programs or applications for portable electronics) for practitioners, organizations, or individuals (e.g., patients). The described features, operations, or characteristics may be combined in any suitable manner in one or more embodiments. The order of the steps or actions of the methods described in connection with the embodiments disclosed may be varied. Thus, any order in the drawings or Detailed Description is for illustrative purposes only and is not meant to imply a required order, nor is every element necessary required in the broadest possible implementations.

Embodiments may include various features, which may be embodied in machine-executable instructions executed by a general-purpose or special-purpose computer (or another electronic device). Alternatively, the features may be performed by hardware components that include specific logic for performing the steps or by a combination of hardware, software, and/or firmware. Any of the various embodiments may include various encryption and/or authentication measures to ensure the security and/or authenticity of the data.

Many of the embodiments described herein may be implemented and/or provided in the form of a computer program product, such as a non-transitory machine-readable medium having stored thereon instructions that may be used to program a computer (or another electronic device such as a controller, processor, or microprocessor) to perform processes and operations described herein. The machine-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of media/machine-readable medium suitable for storing electronic instructions.

The various functional components of the described systems and methods may be modeled as a functional block diagram that includes one or more remote terminals, networks, servers, data exchanges, and software/hardware/firmware modules configured to implement the various functions, features, methods, and concepts described herein. In many instances, each application, embodiment, variation, option, service, and/or another component of the systems and methods described herein may be implemented as a module of a larger system. Each module may be implemented as hardware, software, and/or firmware, as would be understood by one of skill in the art for the particular functionality, and may be part of a larger physical system that may include computer-readable instructions, processors, servers, endpoint computers, and/or the like.

Disclosed herein are embodiments of systems, methods, apparatus, circuits, and/or interfaces. As stated above, the embodiments disclosed herein may be embodied as executable instructions stored on a non-transitory machine-readable storage medium. The instructions may comprise computer program code that, when executed and/or interpreted by a computing device, causes the computing device to implement the processing steps and/or operations disclosed herein. The embodiments disclosed herein may be implemented and/or embodied as a driver, a library, an interface, an application programming interface (API), firmware, Field Programmable Gate Array (FPGA) configuration data, and/or the like. Accordingly, portions of the embodiments disclosed herein may be accessed by and/or included within particular modules, processes, and/or services (e.g., incorporated within a kernel layer of an operating system, within application frameworks and/or libraries, within device drivers, in user-space applications and/or libraries, and/or the like). Alternatively, or in addition, the embodiments disclosed herein may be implemented as particular machine components, which may include, but are not limited to: circuits, processing components, special-purpose processors, general-purpose processors, interface components, hardware controller(s), programmable hardware, programmable logic elements, FPGAs, Application-Specific Integrated Circuits (ASICs), and/or the like.

The embodiments disclosed herein improve the operation of a computing device by, inter alia, enabling coordination between separate, standalone applications operating on the computing device. The embodiments disclosed herein improve the operation of networked computing devices by, inter alia, enabling coordination between separate, standalone applications operating on disparate computing devices. Accordingly, the embodiments disclosed herein may provide additional functionality that does not exist in a general-purpose computing device and/or may improve the operation of the computing device by coordinating the operation of general-purpose applications that do not include coordination-specific functionality. Accordingly, the embodiments disclosed herein may improve the operation of the particular applications operating on the computing device and/or improve the operation of particular applications normally operated on disparate and distinct computing devices.

Additional understanding of the embodiments of this disclosure may be gained by reference to the drawings. Numerous specific details are provided for a thorough understanding of the embodiments described herein. However, those of skill in the art will recognize that one or more of the specific details may be omitted, or other methods, components, or materials may be used. In some cases, operations are not shown or described in detail. For example, well-known features and functions normally employed in other fields of use that are incorporated in the presently described embodiments in new ways are only described to the extent necessary to understand the integration of the features and functions in the respective embodiments of this disclosure.

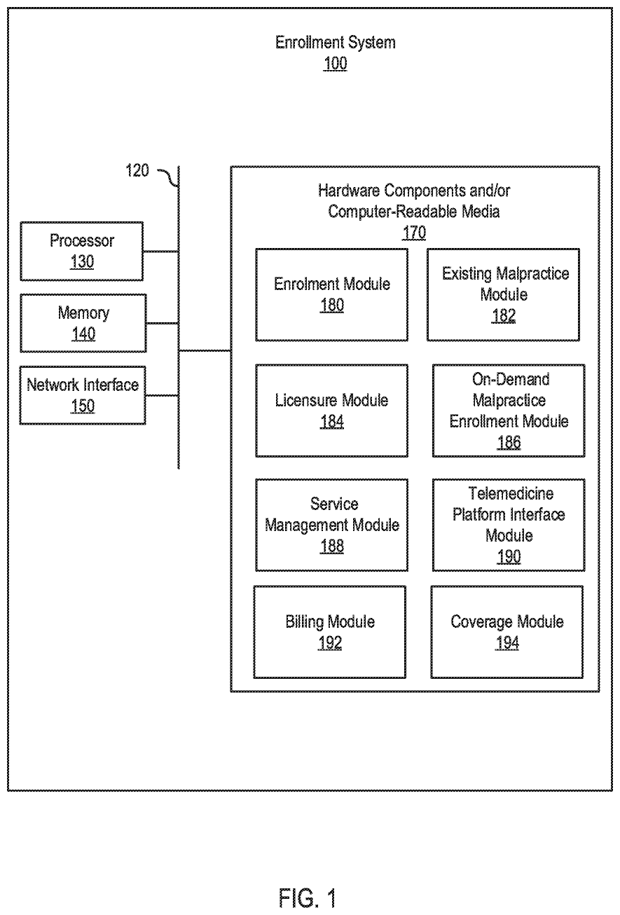

FIG. 1 illustrates an example of an enrollment system 100 to enroll a healthcare practitioner in a telemedicine platform with on-demand per-encounter malpractice insurance coverage. A processor 130 may be connected to a memory 140, a network interface 150, and a computer-readable medium 170 (or comparable hardware components) via a bus 120. Many possible divisions of modules and submodules (or the equivalent subsystems) are possible. However, in the illustrated example, the computer-readable medium 170 includes an enrollment module 180, an existing malpractice module 182, a licensure module 184, an on-demand malpractice enrollment module 186, a service management module 188, a telemedicine platform interface module 190, a billing module 192, and a coverage module 194.

In various embodiments, the enrollment module 180 facilitates the collection of enrollment information. For example, the enrollment module may receive practitioner credentials for enrollment in a telemedicine platform that facilitates various types of telemedicine encounters. Telemedicine encounters may include encounters between the healthcare practitioner and a patient and/or encounters between the healthcare practitioner and another healthcare practitioner (e.g., provider-to-provider consultations). The telemedicine platform may facilitate telemedicine encounters (e.g., consultations, visits, evaluations, examinations, etc.) of various types, including, without limitation, telephone visits, store-and-forward photo visits, and video conferencing visits.

The existing malpractice module 182 (or more generally, an existing coverage module) may facilitate receiving and validating an existing malpractice insurance policy of a healthcare practitioner. For example, the existing malpractice module 182 may request, receive, and/or verify credentials provided by the user relating to an existing malpractice coverage plan that is applicable to in-person encounters between the user and patients.

The licensure module 184 may collect and validate licensure information. For example, the licensure module 184 may request, receive, and/or verify licensure credentials from the user relating to licenses granted to the user authorizing the practice of the user. Such licenses may be granted by medical associates, dental associations, countries, states, cities, counties, or other governmental, regulatory, or trade association entities.

The on-demand malpractice enrollment module 186 may facilitate the offering of, the procurement of, the management of, and/or the calculation of per-encounter malpractice coverage for the practitioner that covers the telemedicine encounters conducted on the telemedicine platform. The pricing may be based on each individual encounter or on sets of encounters. In some instances, the pricing may have maximum fee caps associated with each unique patient, maximum fee caps for each day that telemedicine encounters are used, and/or maximum fee caps charged to the healthcare practitioner in a given time period (e.g., daily, monthly, etc.).

As an example, a healthcare practitioner may have an existing malpractice insurance policy that covers in-person encounters with patients (e.g., consultations, visits, diagnoses, prescription writing, evaluations, recommendations, examinations, and the like). However, the healthcare practitioner's existing malpractice insurance policy may not clearly apply to telemedicine encounters, or even expressly state that it does not apply to telemedicine encounters. The healthcare practitioner may be interested in offering telemedicine offers to patients, but the need to obtain a new malpractice insurance policy may be an impediment or inconvenience.

The presently described systems and methods allow the healthcare practitioner to enroll in an existing telemedicine platform that includes or offers on-demand per-encounter malpractice insurance that applies only to those telemedicine encounters conducted through the telemedicine platform. The on-demand per-encounter malpractice insurance may not apply to any in-person encounters. Instead, the on-demand per-encounter malpractice insurance supplements the practitioner's existing insurance policy(ies). Moreover, the on-demand per-encounter malpractice insurance policy involves minor transaction costs and avoids the possibility that the healthcare practitioner will pay for a policy that is used very little.

The service management module 188 may facilitate the selection of which of a plurality of services the healthcare practitioner wishes to provide via the associated telemedicine platform. For example, the service management module 188 may determine the quantity and type of telemedicine encounters conducted by the user during a time period. The billing module 192 may use this information to bill the user at the end of each time period based on, for example, specific characteristics of the healthcare practitioner, residence location of the healthcare practitioner, residence location of the patient(s), the licenses of the healthcare practitioner (e.g., the fees may be higher for one type of doctor and lower for another type of doctor), subsequent in-office procedures performed on the patient, the quantity of telemedicine encounters conducted during the time period, and/or the type information of each of the telemedicine encounters conducted during the time period.

The telemedicine platform interface module 190 may facilitate communication and interoperation between a dedicated system offering on-demand per-encounter liability (e.g., malpractice) coverage and any of a wide variety of telemedicine platforms, including third-party telemedicine platforms. The on-demand malpractice enrollment module 186 may procure per-encounter liability coverage from third-party insurers and may use different insurers to cover different healthcare practitioners. The coverage module 194 may connect the user to the specific insurer providing the per-encounter malpractice coverage policy (e.g., through a graphical user interface, hyperlinks, portals, chat windows, or the like).

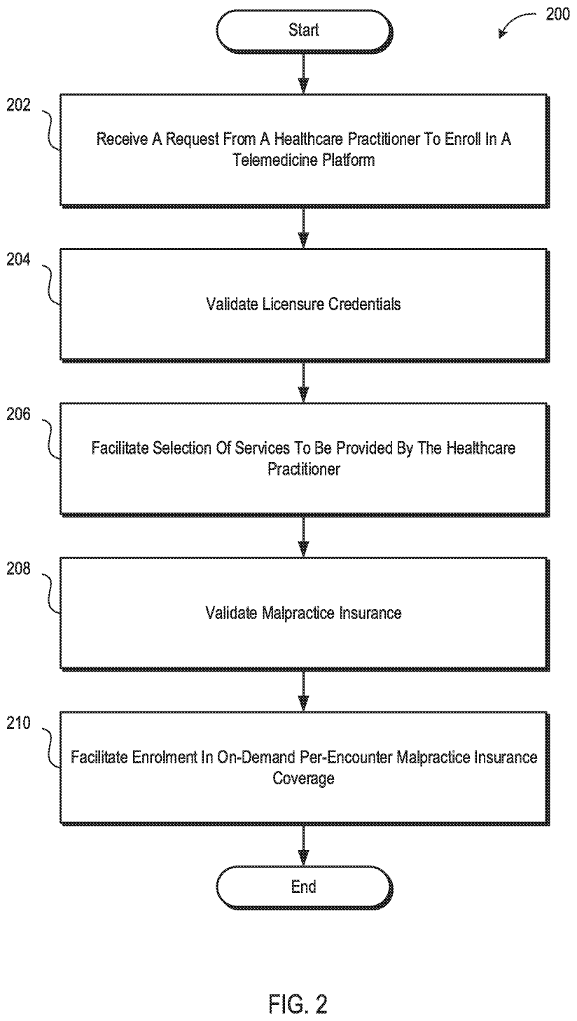

FIG. 2 illustrates a flow chart of a method to enroll a healthcare practitioner in a telemedicine platform, including enrollment in an on-demand per-encounter malpractice insurance coverage program. The enrollment system may receive, at 202, a request from a healthcare practitioner to enroll in a telemedicine platform. The enrollment system may validate, at 204, the licensure credentials. The enrollment system may facilitate, at 206, the selection of services to be provided by the healthcare practitioner.

In some examples, the enrollment system may validate, at 208, any existing malpractice insurance provided by the healthcare practitioner or request validation by a third-party. The enrollment system may facilitate enrollment (e.g., offer, obtain, provide, administer, price, charge for, etc.), at 210, in an on-demand per-encounter malpractice insurance coverage plan or policy. As described herein, the per-encounter malpractice insurance coverage may alternatively be per-visit, per-patient, per-day, per-week, per-month, per-service type, etc.

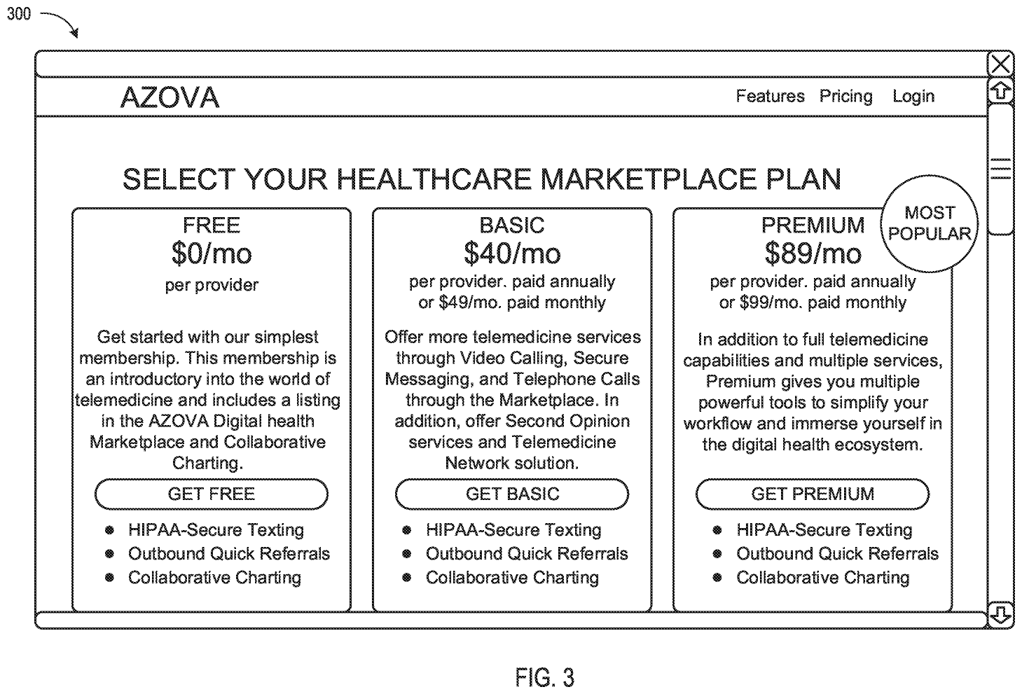

FIG. 3 illustrates an embodiment of an example graphical user interface 300 of an enrollment system to facilitate enrollment in a telemedicine healthcare marketplace plan of a telemedicine platform. The illustrated example includes three different tiers of enrollment in a telemedicine platform associated with the enrollment system.

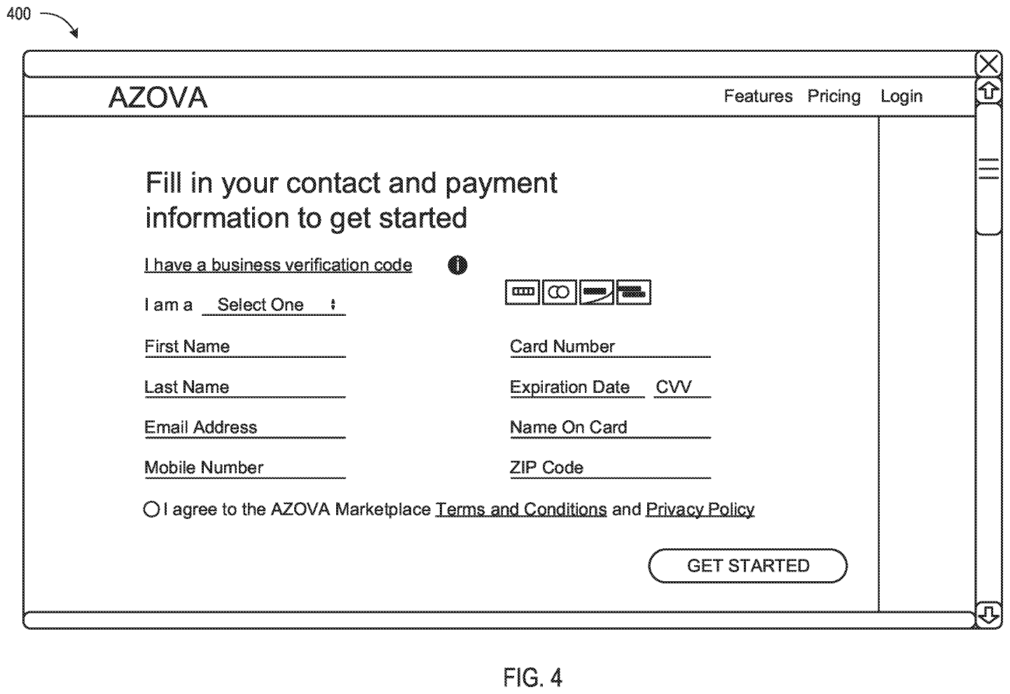

FIG. 4 illustrates an embodiment of an example graphical user interface 400 for collecting enrollment information, including payment information. The enrollment system may validate the personal information and use the billing information to charge for the services provided by the associated telemedicine platform.

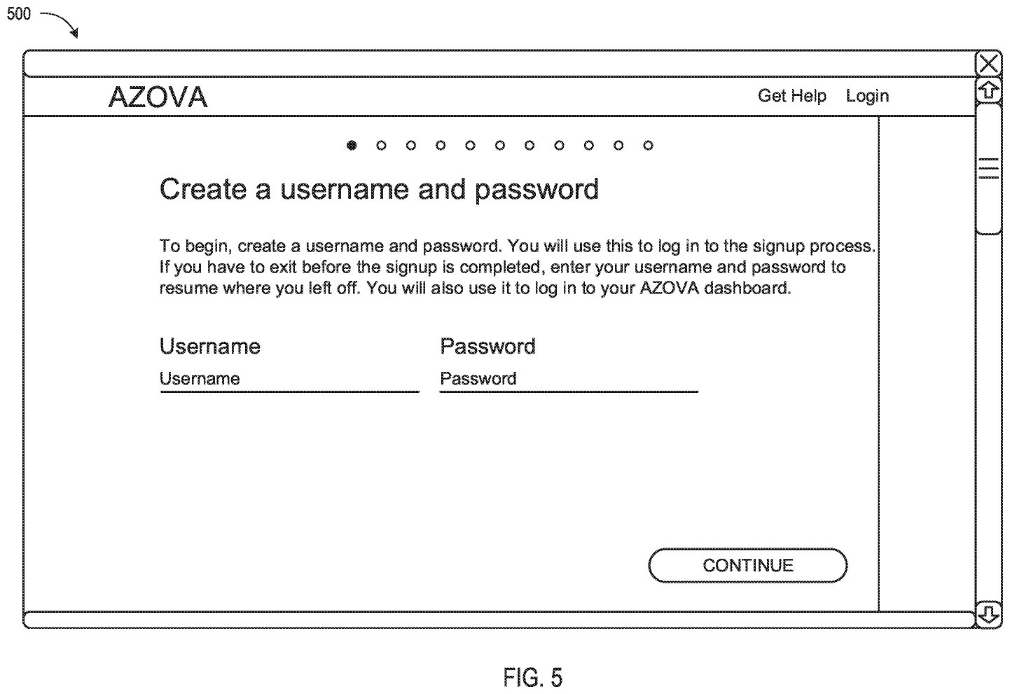

FIG. 5 illustrates an embodiment of an example graphical user interface 500 for creating a username and password for future logins into the telemedicine platform. Any of a wide variety of alternative login credential techniques and approaches may be utilized by the associated telemedicine platform and/or the enrollment system. For example, in some embodiments, blockchain-based credentials may be utilized.

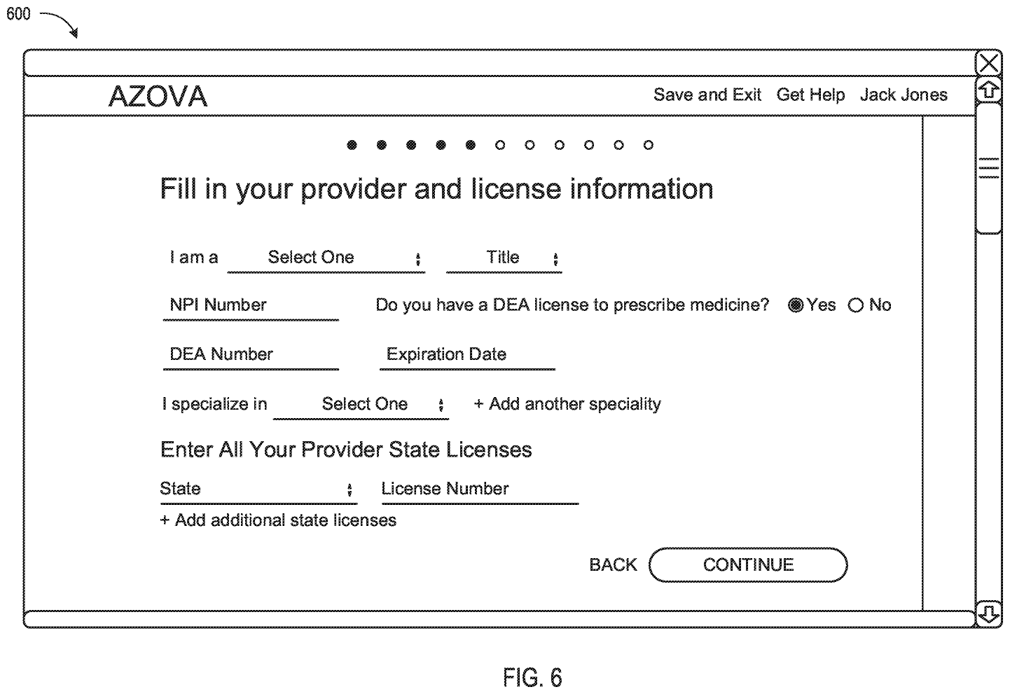

FIG. 6 illustrates an embodiment of an example graphical user interface 600 for a healthcare practitioner to provide license information for validation by the enrollment system. In various embodiments, the enrollment system may be in communication with or include various databases to facilitate validation of the license information provided by the healthcare practitioner.

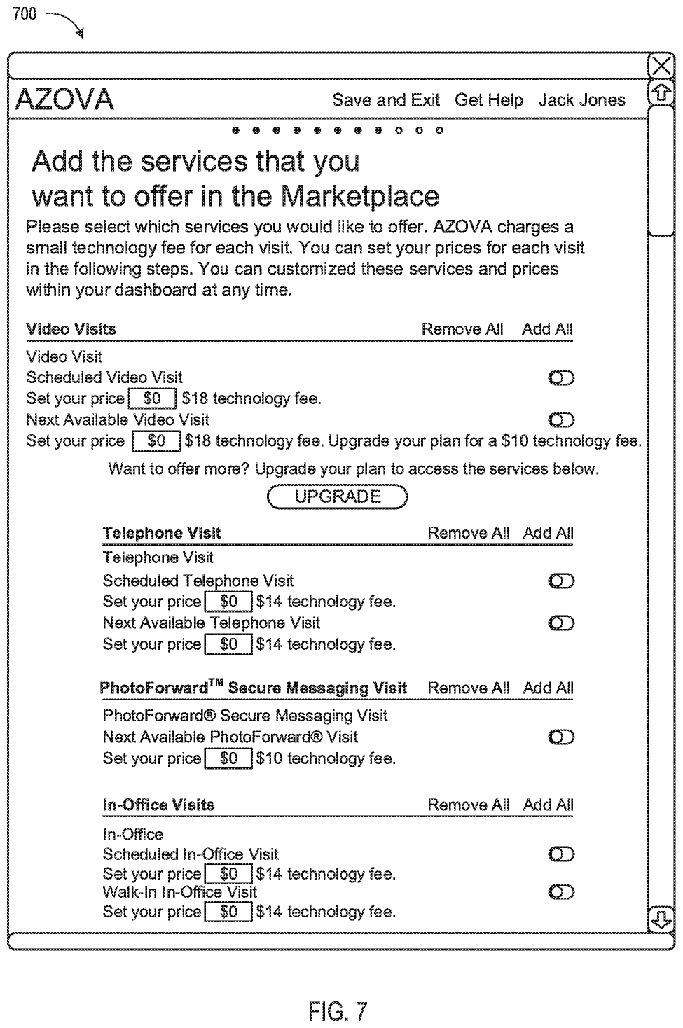

FIG. 7 illustrates an embodiment of an example graphical user interface 700 of the enrollment system to facilitate the selection of services to be offered by the healthcare practitioner via the telemedicine platform. The illustrated examples are merely some of the possible consultation types to be offered via the telemedicine platform by the healthcare practitioner.

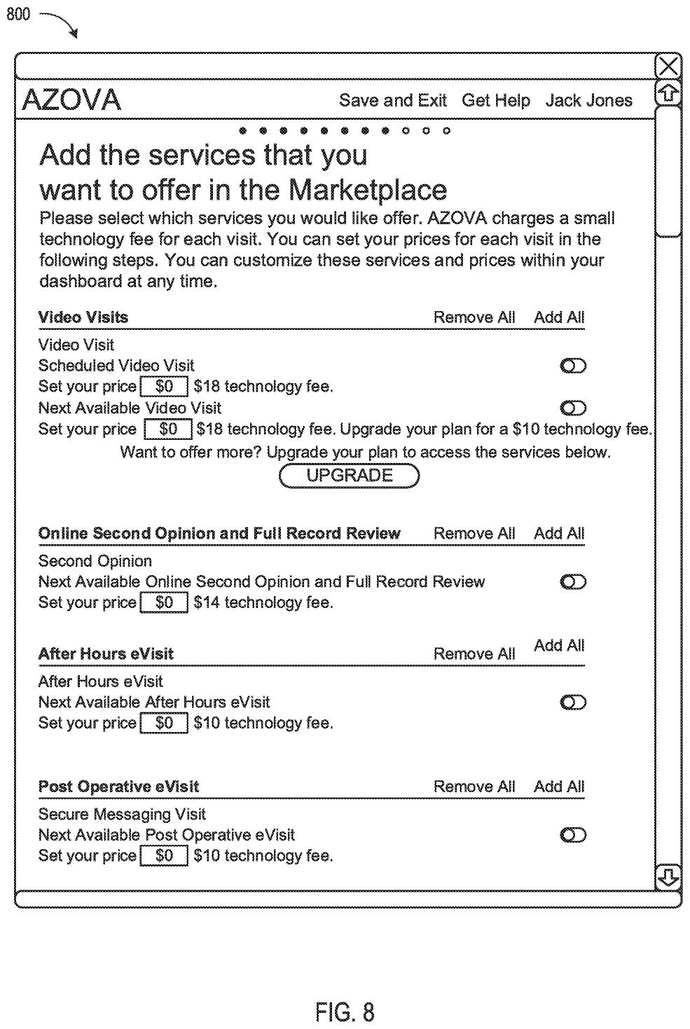

FIG. 8 illustrates an embodiment of an example graphical user interface 800 of the enrollment system to facilitate the selection of additional services to be offered by the healthcare practitioner via the telemedicine platform.

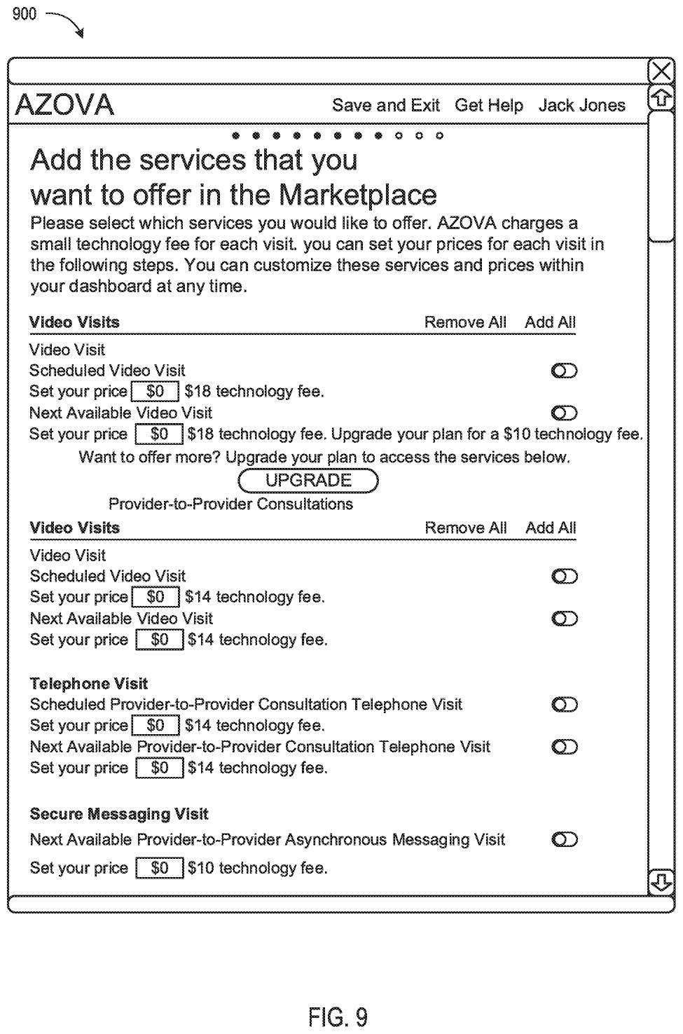

FIG. 9 illustrates an embodiment of an example graphical user interface 900 of the enrollment system to facilitate the selection of additional services to be offered by the healthcare practitioner via the telemedicine platform.

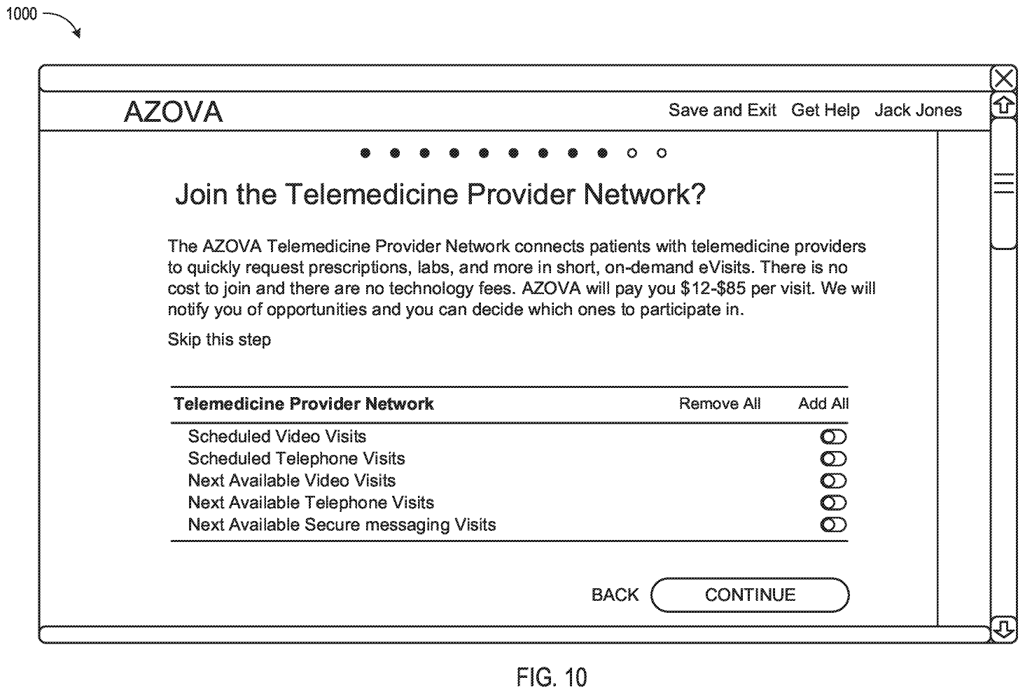

FIG. 10 illustrates an embodiment of an example graphical user interface 1000 of the enrollment system to enable the healthcare practitioner to join a telemedicine provider network of the telemedicine platform.

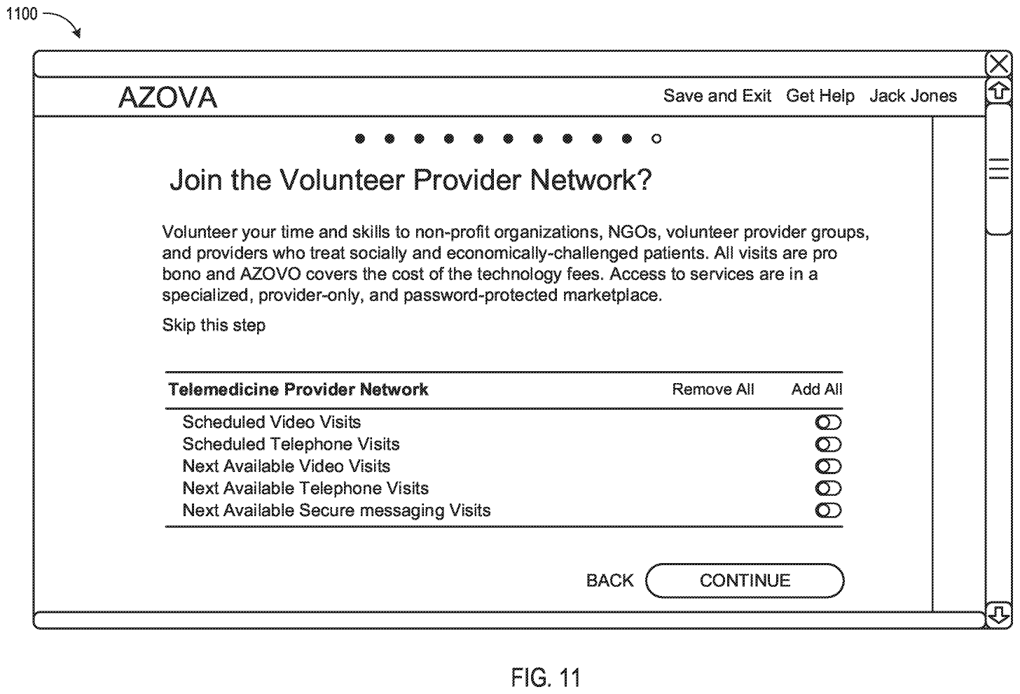

FIG. 11 an embodiment of an example graphical user interface 1100 of the enrollment system to enable the healthcare practitioner to join a volunteer provider network of the telemedicine platform.

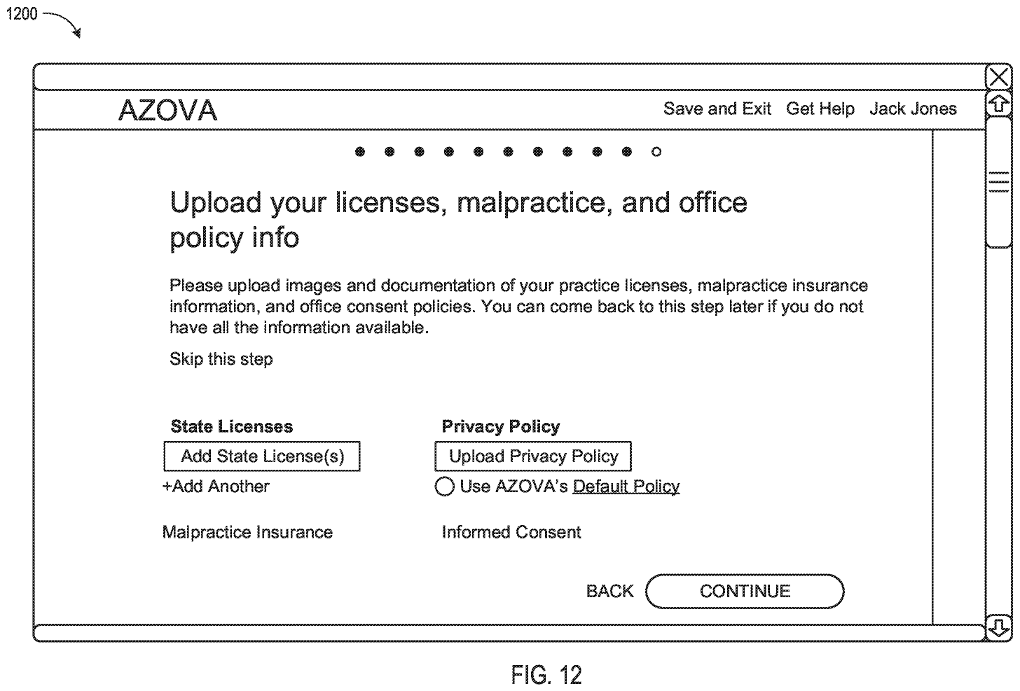

FIG. 12 illustrates an embodiment of an example graphical user interface 1200 of the enrollment system to receive state licenses and malpractice insurance information.

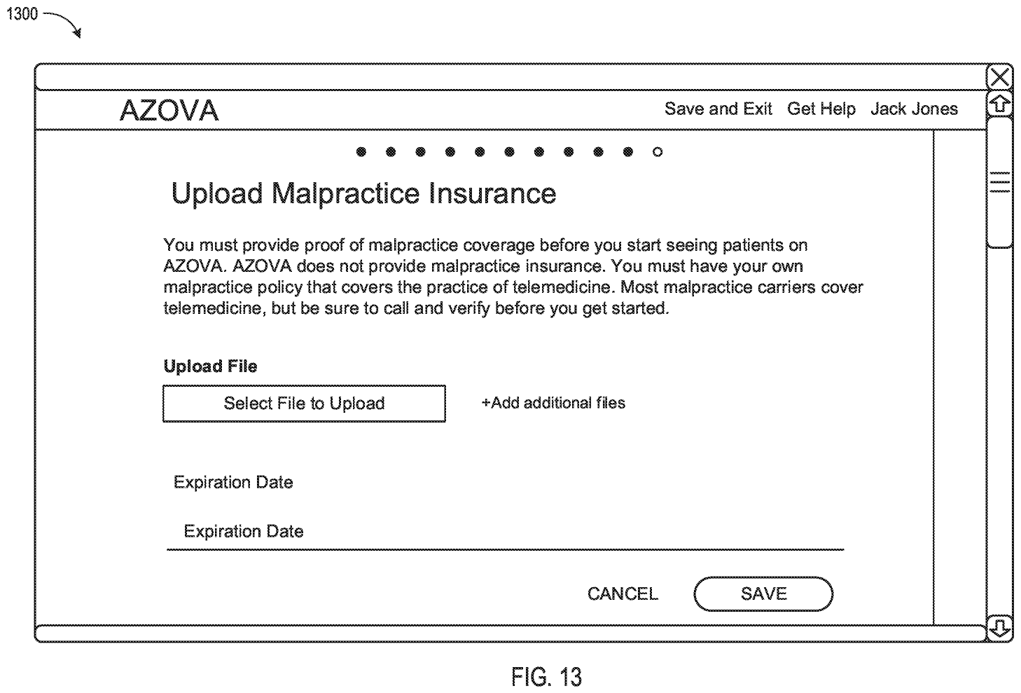

FIG. 13 illustrates an embodiment of an example graphical user interface 1300 of the enrollment system to prompt the healthcare practitioner to enter existing malpractice insurance information. Again, the enrollment system may be in communication with and/or include various databases to facilitate the validation of existing malpractice insurance information provided by the healthcare practitioner.

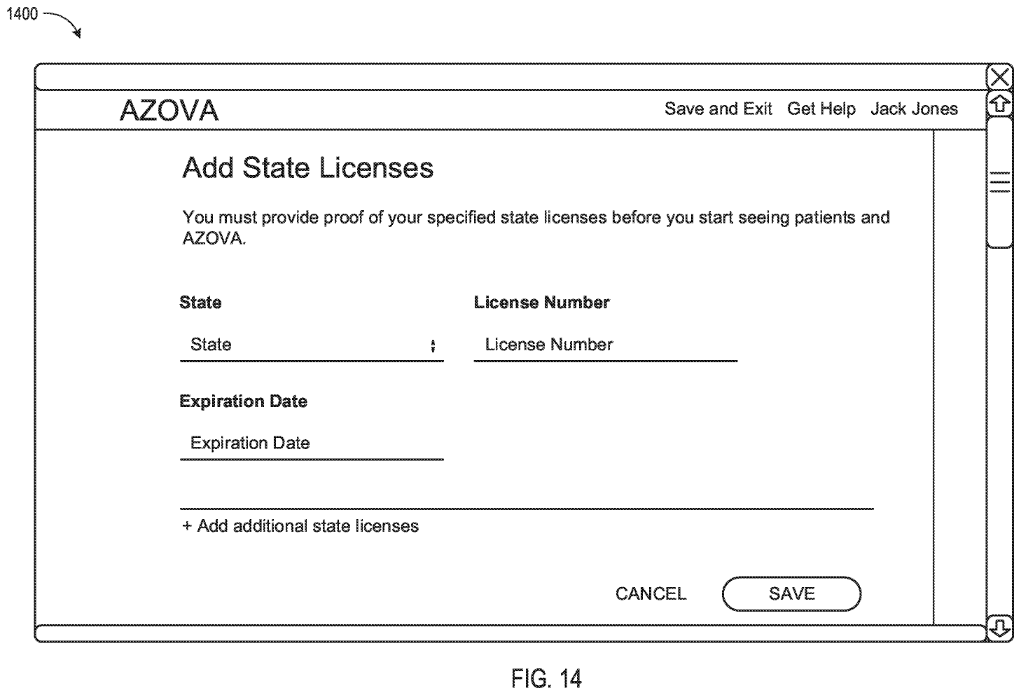

FIG. 14 illustrates an embodiment of an example graphical user interface 1400 of the enrollment system to prompt the healthcare practitioner to enter state license information for validation by the enrollment system.

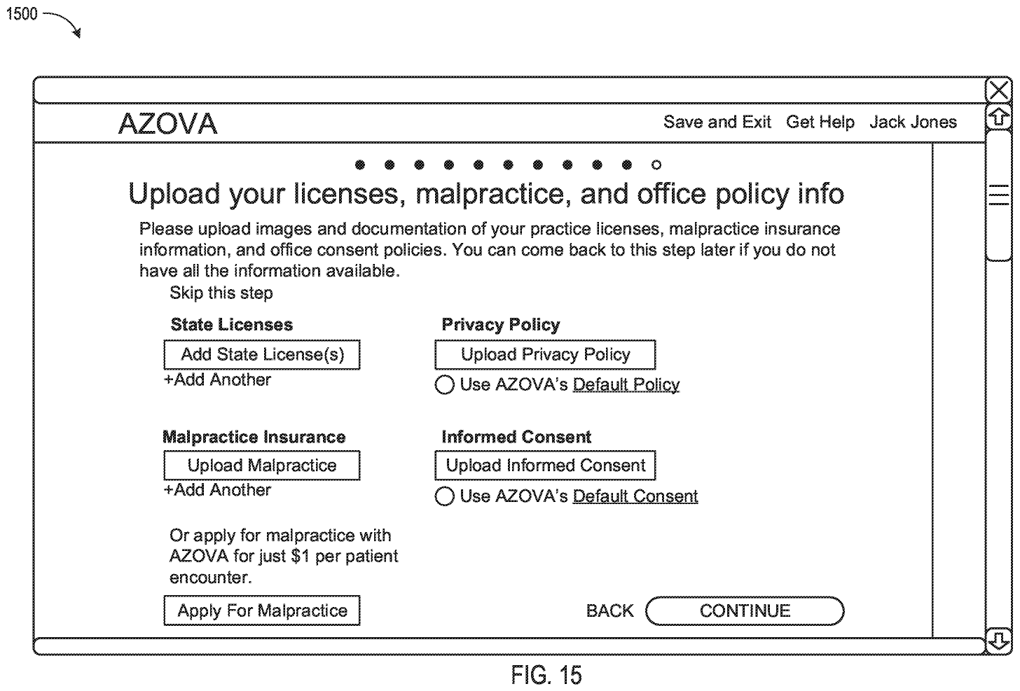

FIG. 15 illustrates an embodiment of an example graphical user interface 1500 of the enrollment system to invite the healthcare practitioner to enroll or apply for malpractice insurance. The on-demand per-consultation malpractice insurance may be the only malpractice insurance the healthcare practitioner has, replace an existing malpractice insurance policy of the healthcare practitioner, or supplement an existing malpractice insurance policy of the healthcare practitioner.

The on-demand per-consultation malpractice insurance may be on-demand in the sense that it is available in real-time during enrollment in the telemedicine platform and/or because the malpractice insurance is only applicable to consultations/visits conducted through the telemedicine platform associated with the enrollment system (i.e., not the healthcare practitioner's normal, in-office consultations/visits). Accordingly, the healthcare practitioner may be billed for the malpractice insurance on a per-consultation basis.

For example, the healthcare practitioner may have a malpractice insurance policy that covers her normal medical practice, including in-office visits. The healthcare practitioner may be interested in providing telemedicine services even though her existing malpractice insurance policy does not cover telemedicine visits and/or would incur additional expenses and/or be difficult or troublesome to update. The enrollment system described herein allows the healthcare practitioner to, for example, enroll in supplemental on-demand per-consultation malpractice insurance that will provide liability coverage for the healthcare practitioner's telemedicine consultations. The healthcare practitioner may pay for such insurance on a per-visit basis or other periodic basis associated with the number of actual visits, actual patients, actual consultation types, and/or prescriptions written (e.g., $1 per visit, $10 per patient, $100 for the first 200 patients, $1 per prescription written, $20 per day, and/or combinations thereof on a one-time or periodic basis).

The various insurance policy options may be fully or partially customized based on the particular healthcare practitioner's practice and/or selected services to offer via the telemedicine platform. Any of a wide variety of health and wellness platforms and/or services may be integrated as part of the enrollment process offered by the enrollment system

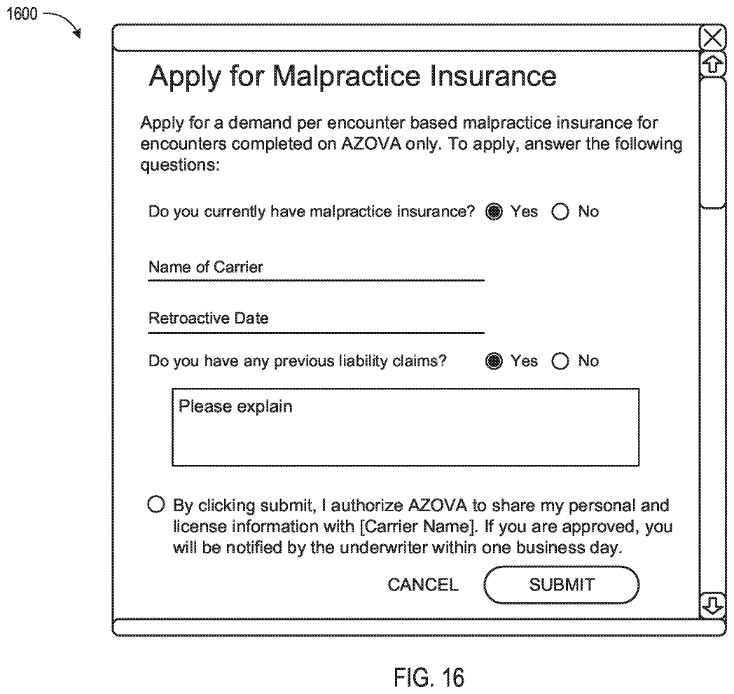

FIG. 16 illustrates an embodiment of an example graphical user interface 1600 of the enrollment system requesting information as part of a malpractice insurance application. As noted in the illustrated example, the application for malpractice insurance may be submitted to an underwriter of one or more specific insurance carriers for final approval. In some examples, subsequent communication regarding the malpractice insurance policy may be solely between the insurance carrier and the healthcare practitioner. In other examples, the enrollment system and/or associated telemedicine platform may act as an intermediary between the insurance carrier and healthcare practitioner.

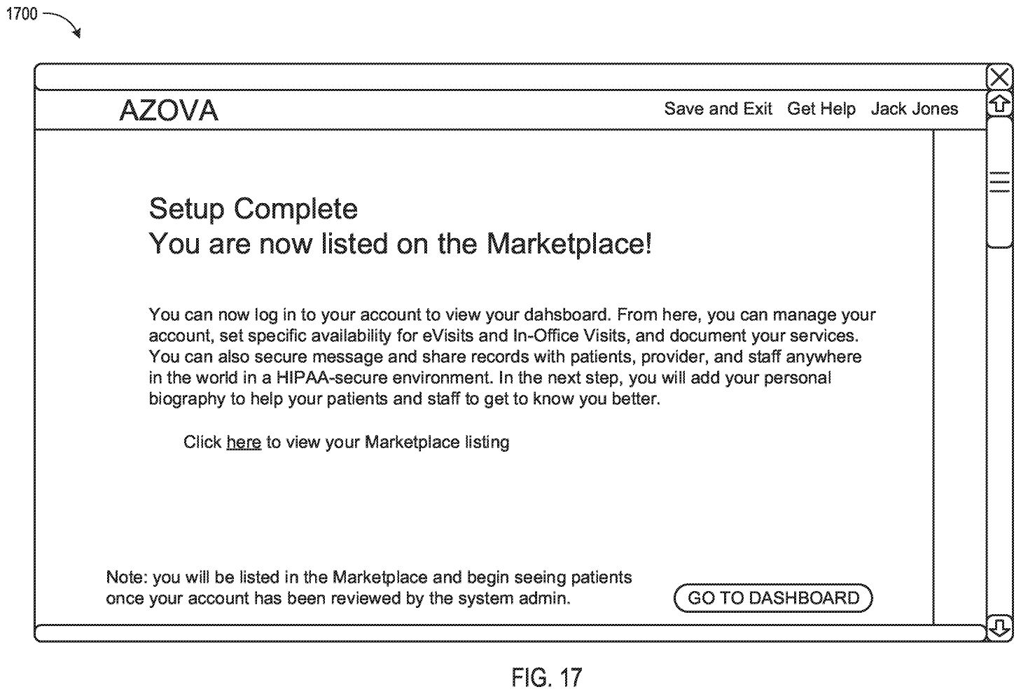

FIG. 17 illustrates an embodiment of an example graphical user interface 1700 of the enrollment system confirming enrollment.

Many changes may be made to the details of the above-described embodiments without departing from the underlying principles of the present disclosure. Moreover, all combinations and permutations of each of the embodiments and functions described herein and claimed below are contemplated and may be useful in a particular application or situation.

What is claimed:

1. An enrollment system, comprising:
a processor; and
a non-transitory computer-readable medium with instructions stored thereon that, when executed by the processor, cause the enrollment system to:
render, for display on an electronic display, an enrollment graphical user interface that prompts a user to enroll in a telemedicine platform, wherein the telemedicine platform facilitates telemedicine encounters between the user and patients;
receive a request from the user to enroll in the telemedicine platform;
generate a per-encounter liability coverage offering for the user to cover the telemedicine encounters, wherein the per-encounter liability coverage offering is based, at least in part, on a characteristic of the user; and
render, for display on the electronic display, a coverage graphical user interface that prompts the user to enroll in the per-encounter liability coverage.

2. The system of claim 1, wherein the instructions, when executed by the processor, further cause the enrollment system to:
render, for display on the electronic display, a validation graphical user interface that prompts a user to enter credentials of an existing liability coverage plan applicable to in-person encounters between the user and patients;
receive the credentials of the existing liability coverage plan; and
validate the credentials of the existing liability coverage plan.

3. The system of claim 1, wherein the enrollment graphical user interface further prompts a user to select between at least two different service tiers of the telemedicine platform.

4. The system of claim 1, wherein the telemedicine platform facilitates telemedicine encounters between the user and other healthcare practitioners.

5. The system of claim 1, wherein the per-encounter liability coverage offering includes a first pricing model for telemedicine encounters between the user and patients and a second pricing model for telemedicine encounters between the user and other healthcare practitioners.

6. The system of claim 1, wherein the per-encounter liability coverage offering comprises an on-demand per-encounter medical malpractice insurance coverage offering.

7. The system of claim 1, wherein the characteristic of the user on which the per-encounter malpractice coverage offering is, at least in part based, comprises one or more of: a residence of the user, residences of the patients, and the licensure credentials of the user.

8. The system of claim 1, wherein the characteristic of the user on which the per-encounter malpractice coverage offering is, at least in part based, comprises a ratio of in-person encounters conducted by the user to telemedicine encounters conducted by the user.

9. The system of claim 1, wherein the instructions, when executed by the processor, further cause the enrollment system to:
render, for display on the electronic display, a selection graphical user interface that prompts a user to select one or more encounter types from a set of encounter types available via the telemedicine platform; and
receive a selection by the user of one or more of the available encounter types,
wherein the per-encounter liability coverage offering is further based, at least in part, on the encounter types selected by the user.

10. The system of claim 9, wherein the set of encounter types includes at least some of: telephone visits, store-and-forward photo visits, and video conferencing visits.

11. A per-encounter risk management system, comprising:
an enrollment module to receive practitioner credentials for enrollment in a telemedicine platform, wherein the telemedicine platform facilitates telemedicine encounters;
an on-demand malpractice enrollment module to obtain per-encounter malpractice coverage for the practitioner to cover the telemedicine encounters, wherein pricing of the per-encounter malpractice coverage is based, at least in part, on a characteristic of the user; and
a billing module to bill the user a fee for the per-encounter malpractice coverage based on the number of telemedicine encounters conducted by the user via the telemedicine platform.

12. The system of claim 11, further comprising a service management module to:
determine quantity and type information for telemedicine encounters conducted by the user during a time period,
wherein the billing module is configured to bill the user at the end of each time period based on:
(i) the quantity of telemedicine encounters conducted during the time period, and
(ii) the type of information of each of the telemedicine encounters conducted during the time period.

13. The system of claim 11, an existing coverage module to:
receive credentials from the user relating to an existing malpractice coverage plan applicable to in-person encounters between the user and patients; and
validate the credentials relating to the existing malpractice coverage plan.

14. The system of claim 11, further comprising a licensure module to:
receive licensure credentials from the user relating to licenses granted to the user authorizing the practice of the user; and
validate the licensure credentials.

15. The system of claim 11, further comprising a coverage module to:
connect the user to an insurer providing the per-encounter malpractice coverage.

16. The system of claim 11, wherein the pricing of the per-encounter malpractice coverage is the same for each type of telemedicine encounter available via the telemedicine platform.

17. The system of claim 11, wherein the telemedicine platform facilitates telemedicine encounters between the user and patients and between the user and other healthcare practitioners, and wherein the per-encounter malpractice coverage covers each type of telemedicine encounter available via the telemedicine platform.

18. A method for insuring a healthcare practitioner, comprising:
confirming that a healthcare practitioner is licensed;
confirming that the healthcare practitioner has an existing malpractice insurance policy that covers in-person patient treatment;
calculating an insurance fee to be charged for each set of telemedicine consultations provided by the healthcare practitioner via a telemedicine platform;
providing the healthcare practitioner with malpractice insurance coverage for telemedicine consultations conducted by the healthcare practitioner via the telemedicine platform;
determining the number of telemedicine consultations provided by the healthcare practitioner via the telemedicine platform during a time period; and
billing the healthcare practitioner based on the determined number of telemedicine consultations provided during the time period.

19. The method of claim 18, wherein the insurance fee to be charged for each set of telemedicine consultations comprises a fee for each individual telemedicine encounter.

20. The method of claim 18, wherein the insurance fee to be charged for each set of telemedicine consultations is based, at least in part, on one or more of the following:
a maximum fee cap associated with each unique patient,
a maximum fee cap for each day, and
a maximum fee cap for the time period.
